(19) United States
(12) Patent Application Publication
(10) Pub. No.: US 2023/0016388 A1
(43) Pub. Date: Jan. 19, 2023

(54) AUTONOMOUS UVC DISINFECTION ROBOT FOR MASS TRANSPORTATION

(71) Applicants: Kristopher Douglas Rupay, Ottawa (CA); Arash Mahinakbarzadeh, Ottawa (CA); Payam Peter Bahraini, Ottawa (CA)

(72) Inventors: Kristopher Douglas Rupay, Ottawa (CA); Arash Mahinakbarzadeh, Ottawa (CA); Payam Peter Bahraini, Ottawa (CA)

(21) Appl. No.: 17/373,377

(22) Filed: Jul. 12, 2021

(51) Int. Cl. A61L 2/10; A61L 9/00; A61L 9/20

(52) U.S. Cl. CPC A61L 2/10; A61L 9/00; A61L 9/20; A61L 2202/11; A61L 2202/16; A61L 2202/14; A61L 2209/11; A61L 2209/12

(57) ABSTRACT

The invention is an air and surface disinfection robot designed to autonomously move through incredibly narrow spaces, specific to mass transportation vehicles such as aircraft, trains, busses, or ferries. The robot includes modes to operate in the spaces associated with transportation, such as waiting areas, bathrooms and narrow entry and exit ways.

In the transportation industry, turn around time (the time between two planned trips), is often incredibly short. Existing designs do not allow for a fast enough loading, setup and run time. This invention has been designed to optimize the portability, maneuverability, connectivity (data) and ease of use from other devices designed to disinfect such vehicles.

The invention uses UVC (germicidal) lamps, a proven technology for disinfecting pathogens. The design has been optimized by using a unique open cage design, so the light will transmit from all directions with minimal absorption to components on the device. This in combination with the lamp pattern and the robotics results in a very precise UVC light emission required for adequate, consistent disinfection of the surfaces and the air of mass transportation vehicles.

Finally, the invention consists of real time data streaming and remote operation as data is published through a connected cloud service and then onto the subscribed users.

### BACKGROUND

Certain pathogens (specifically viruses and bacteria) have the potential to create a pandemic. These pathogens are often spread by the movement of humans throughout the world. With a continued increase and interest in travel, the threat of spreading deadly pathogens globally continues to rise.

Both passengers and staff of mass transportation vehicles can be the primary carriers and spreaders of these pathogens. Spread can occur on surfaces and in the air of these tight enclosed spaces, specifically that of an aircraft, train, bus, ferry and even the bridges (ramps) and waiting areas leading to such transportation vehicles.

Our invention has been designed to quickly, efficiently, and easily inactivate the DNA and RNA of pathogens on surfaces and in the air, minimizing the spread between passengers and staff between departure and destination locations.

While ultraviolet (UV) light within the UVC spectrum has been used on aircraft and in many other fields (medical, dental, food and beverage etc.), our design has a unique radiation pattern to quickly hit all high touch surfaces of these tight spaces. The invention has been designed to be portable, light weight and easily transportable up stairs and through other tight spaces.

The lamps have been placed in a configuration that allows for light to radiate from all lamps in all directions through an open cage. Combined with this unique radiation pattern, an element of robotics control has been combined to remove the human error when disinfecting through more common manual methods (UVC or Chemical).

In the mass transportation industry, time and data are extremely valuable. The device has been designed to be simple to move, load on board, deploy/operate, and disinfect all while providing a wealth of data throughout the operation. The device has both Wi-Fi and cellular connection ensuring it remains connected throughout its use and while charging. The data on board the invention is streamed in real-time providing valuable information that can be shared amongst operators and other subscribed users.

### SUMMARY

This invention was designed to disinfect the air and high touch surfaces of tights spaces of mass transportation vehicles and related spaces. Although, such devices exist the focus of improvement has been placed on UV efficiency, portability, reduction of time to disinfect, automation and real-time connectivity.

Distinguishing features of the invention include:
1) Efficiency: A unique vertical UVC lamp and open cage configuration has been designed to minimize light absorption, maximize light emission, radiate in all directions, and require zero mechanical adjustments and/or human intervention (i.e., no expandable arms) prior and while in operation.
2) Maintainability: A unique custom “X” design has been used for quick lamp change and to allow for any physical energy (from bumps during transportation) to be absorbed by the device and not to the lamps.
3) Portability: Dimensions, weight and handle placements have been uniquely designed to easily fit through the required tight spaces of mass transportation vehicles (doors, aisles, lavatory, cockpit etc) with the ability to easily load/off-load from the vehicles without the need for a lift or ramps. This allows for incredibly fast operating times, ensuring quick turn-times and on time performance.
4) Autonomous: Several autonomous movement algorithms have been invented to automatically detect the aisle width, detect the direction to move, navigate through the aisles and transition between aisles without selecting any pre-configured settings or the need of human intervention (again reducing operation time).
5) Stationary: The design includes the ability to disinfect smaller enclosed spaces without any movement from the motors. This is to cover disinfection of other spaces (washrooms, lunchrooms, offices, waiting areas) all with the same device.
6) Connectivity: The device remains connected (both Wi-Fi and cellular) prior, during and after operation, providing real time data for statistics, status, remote dashboards, and maintenance. The connection enables, remote operation through a personal computer, laptop, tablet, or mobile device as a secondary option for control.

### BRIEF DESCRIPTION OF DRAWINGS

FIG. 1: An isometric view of the invention.

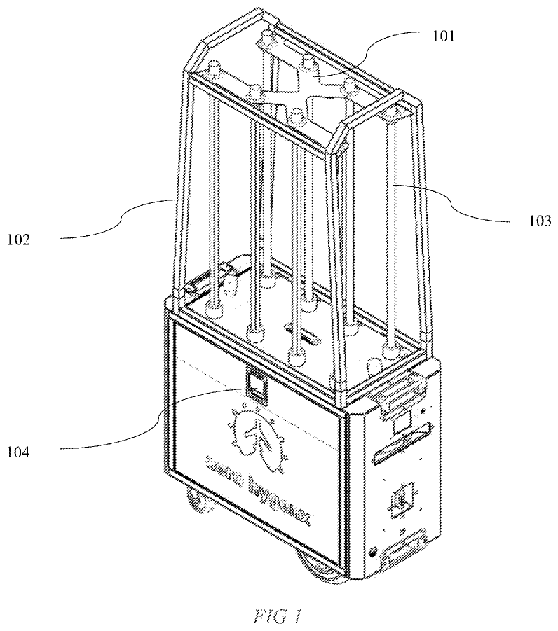

FIG. 2: Front view of the invention. Showing the narrow width, the touch screen(s) to start and the handle placements (top and bottom corners of base).

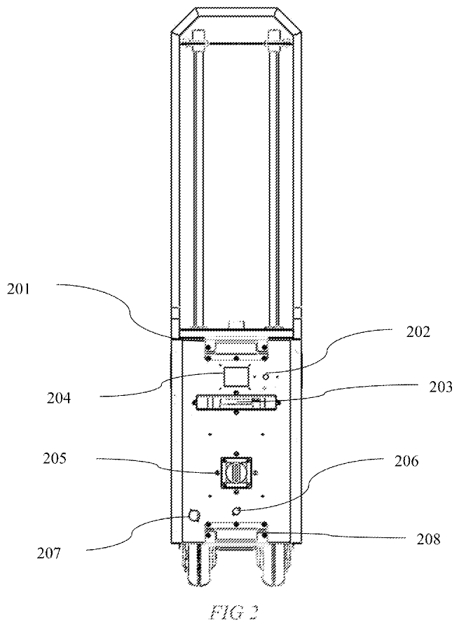

FIG. 3: Rear view of the invention. Showing the narrow width, the touch screen(s) to start and the handle placements (top corner and middle of the base).

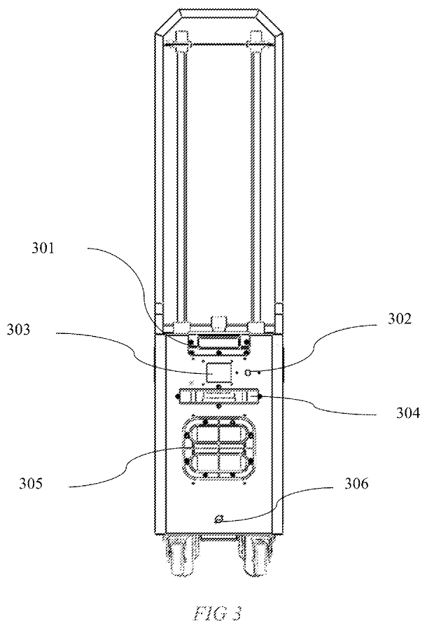

FIG. 4: Top-down view of the invention, showing the placement of the lamps and the top cage design.

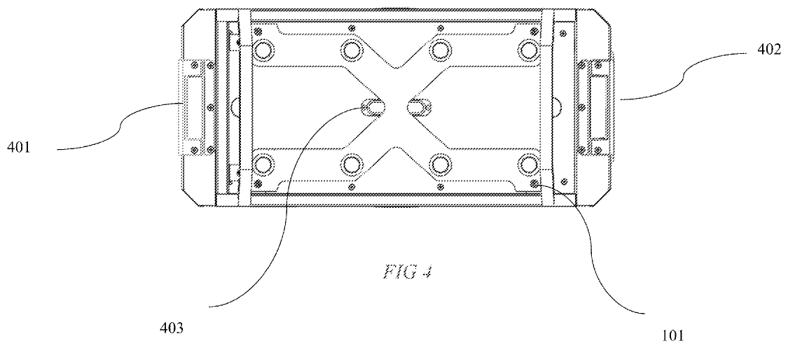

FIG. 5: Side view of the invention, with the easy access panel removed.

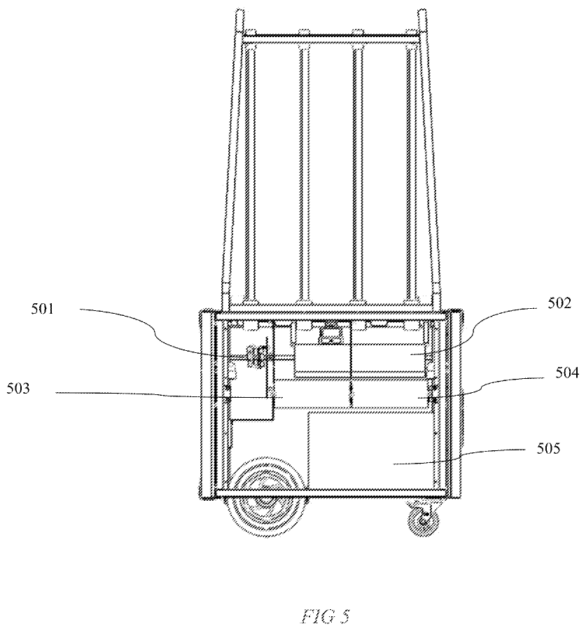

FIG. 6: Bottom view of the invention showing the placement of the drive wheels and the castors.

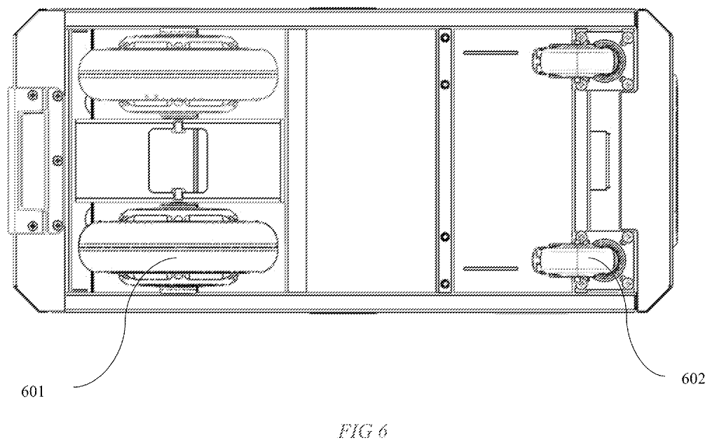

### DETAILED DESCRIPTION

Our invention brings together many technologies to ensure the disinfection of tight spaces, specifically those of mass transportation vehicles. The invention focuses on UV efficiency, device portability, custom autonomous movement, maintainability, and connectivity.

The disinfection technology that is being utilized is germicidal UVC electromagnetic radiation lamps. Germicidal lamps 103 produce a unique wavelength of light, know to scientists for over a century. It yields incredibly high disinfection results when the correct dosage of light reaches a surface or passes through open space. Wavelengths in this spectrum of light transmit right to the pathogens DNA or RNA.

When the light reaches the DNA or RNA it alters this genetic material, preventing these cells from ever replicating again, thus stopping the spread of the pathogen.

UVC is known to have a very low reflectivity off nearly all surfaces. To succeed in the mass transportation industry, disinfection efficiency is imperative. The invention has been designed without any vertical reflective components (often found in the centre of devices, between lamps), utilizing an open cage design 102. The germicidal lamps 103 radiate light in all directions around the cylinder of every lamp (360 degrees), and in all directions up and down (180 degrees) along the length of the lamp. The open cage design 102 enables minimal light absorption, thus allowing the maximum amount of light to radiate away from the device. By maximizing the radiation of light, the design can now reduce the overall footprint of the design and reduce the required batteries 503 to power the device (discussed further below).

Vertical lamps 103 have been carefully chosen on the invention to radiate the required light through the open cage 102, while moving at a quick (but safe) speed (autonomous speed discussed further below) through the tight spaces.

The lamps 103 are in a fixed position and are not required to move prior to starting the operation. This again, aids with the time for a quick setup and start of the robot.

Maintaining the lamps has been designed to be incredibly easy, yet at the same time protect the lamps. The lamps slide from the top down through a custom “X” pattern lamp guide 101. This custom lamp guide uses a high heat resistant grommet allowing the lamps to slide through, and to move ever so slightly. This tolerance between the grommets and the lamps removes potential stress on the lamps that could be caused from the device getting bumped around during transportation.

Portability is paramount to the design, as handling of the device between disinfection locations must be incredibly easy. The width of the device has been designed to be incredibly narrow to fit through the tight spaces of aisles and doorways.

Handles have been placed in ergonomic positions 201, 208, 208, 301 to allow for easy movement up and down stairs. The rear of the device was designed to move up stairs first, with handles placed at the top 301 and in the middle 305. The front of the device has the handles placed on the upper 201 and lower corners 208 for the operator on the lower position of the stairs.

Nearly every component on the device is made of aluminium for light weight (again aiding to portability). Aluminum is a softer metal than most, thus strategic single, double, and even triple bends are used to ensure the required strength requirements were met.

A single battery 505 design is used and placed at the lowest point of the device. This was intended to ensure a very low centre of gravity on the device. The battery selected is a trade secret, but it is a battery known to have light weight properties, operate in a very wide range of temperatures (−40 C to +70 C), have a consistent voltage throughout operation, is safe to travel with, can quickly be charged and has an incredibly long-life span (more than 2000 full cycles).

A drive motor is often the heaviest component of a moving mechanical device. To maintain a very low weight, to help again with portability, light weight electromagnetic hub motors 601 were selected and paired with light weight castors 602 to keep weight and complexity to a minimum.

Being autonomous was paramount to ensure disinfection consistency and repeatability. Many autonomous algorithms were used or developed to control the gentle movement of the electromagnetic hub motors through the tight spaces.

Distance proximity sensors were placed at strategic locations on the front 203, 206 and rear of the device 304, 306. These sensors provide the algorithms with essential data to navigate the device. The custom algorithms working in parallel are a part of this patent, but the exact algorithms are currently trade secrets and will be presented as a separate design patent. These algorithms include:
1) Automatically detect the width of the aisle.
2) Detection of an aisle (from a galley etc.)
3) Automatically keep the device centred while moving through an aisle.
4) Automatically shift if the aisle shifts (i.e., Transition from one cabin to another).
5) Move parallel to a single wall (to the left or right), when an open space is present on the opposite side.
6) Keep the device centred while moving through open spaces.
7) Automatic speed control to ensure the required dosage of light reaches all targeted air and surfaces.
8) Detection of the end of the aisle.
9) Detection of the end of a disinfection cycle.

All algorithms work together to ensure the correct UV light dose is applied by controlling the speed of the electromagnetic drive wheels 601.

To start the autonomous movement a single or double pass can be selected from the touch screens on the front 204 or the rear 303. The single pass will start by automatically detecting the direction to move and will stop when it reaches the end of the selected aisle. A single pass is to allow operators for cabin configuration changes between passes (i.e., Tray tables up on single pass one and down on single pass two). If a double pass is selected the cabin configuration will remain the same.

When running a device such as this, safety is a major part of the autonomous design. The design uses many of the same sensors 202, 203, 206, 302, 304, 306 that navigate the robot, to detect and stop when the following occur:
1) Object detection that may impede the movement.
2) Human detection should a human(s) come too close to the device.
3) Wall and Seat detection to ensure a minimal buffer is maintained.
4) No-movement detection should the device become stuck.
5) Sensor failure should any of the guidance sensors fail.

Finally, as a part of the autonomous movement, and to maintain quick start times, a single touch (press once only) approach was taken with the design. Control can be made from the two touch screens 203, 303 and remotely through a custom app. With the algorithms listed above, the device can navigate down virtually any tight space, aisle, or hallway. The requirement to enter the type of vehicle has been completely removed, again saving operator start and operating time.

Included with the invention is a “stationary disinfection mode”. This allows for a zero-movement disinfection of virtually any space. This is a value-added feature for operators, enabling them to disinfect washrooms, offices, closets, lunchrooms, and other tight closed spaces either on or off the vehicles. Similar to starting the autonomous movement, a single touch 204, 303 starts the stationary mode of operation.

In addition to the automated safety features, manual safety features have been included within the design. Two emergency stop buttons 401, 402 have been placed at either end of the device. When pressed these buttons disable the drive motors 601 and the UVC electronics 502, 503 all while maintaining connectivity to the control board 501.

While transporting the device a master power switch 205 has been included in the design that physically disconnects the battery from all electronics. This is often a requirement when shipping the device with the battery 505 stored within.

Connectivity and the data that is exchanged is a major part of our invention design. The device has been designed to communicate primarily through wireless communication, but it also has a cellular connection should wireless communication is not available 501. Trade marked the “HygenX Stream”, this connection enables the following:
1) Real Time Data Exchange.
2) Remote Control of device.
3) Remote Health Monitoring.
4) Remote software updates.
5) Remote adjustments for custom movement scenarios.
6) Secure shell communication.

In the world of IoT and mass transportation, it is paramount that the design includes an API to securely share real time data from the devices. The robotic and light-based data can be accessed “raw” for health monitoring or is turned into essential statistical based data for subscribed users. This data includes, but is not limited to:
1) Disinfection Status (Percentage complete)
2) Lifetime usage.
3) UVC exposure of a single space (i.e., Aircraft fin)
4) Number of seats disinfected.
5) Disinfections per day.
6) Predictive maintenance (i.e., Expecting a lamp degradation).
7) Time savings by using device compared to other devices.

This information, provided through the Hygenx Stream API, is intended for operators where time is critical or to passengers/transit riders looking for confidence that their mass transportation vehicle has been disinfected to the intended level.

The HygenX Stream allows operators to control their device(s) from a central control centre or personal device (personal computer, tablet, or mobile device). Operators can view all of their devices, select the device of interest and control with all of the same buttons that are located on the local physical touch screens 204, 303 of the device.

Support and maintenance are paramount to time critical operations, thus having the ability to update software, monitor system status and remote directly to the device (using secure shell) is essential. The HygenX Stream comes equipped with all of this.

With reference to the figures, the following table describes the key hardware components within the invention.

| | Name | Description |
|---|---|---|
| 101 | Lamp Guide | A custom lamp guide system that enables protection to the lamps and |
| | | allows for easy installation of the lamps. |
| 102 | Protective Cage | The open cage is required to protect the UVC lamps from impact from |
| | | potential large objects while moving the device from different |
| | | locations. It is meant to be completely open side to side, to minimize |
| | | UVC absorption and to maximize the UVC output. |
| 103 | UVC Lamps | The lamps have been designed to ensure adequate UVC dosage is |
| | | received within the time of the device passing. This can be |
| | | accomplished by many different UVC producing technologies (such as |
| | | Mercury, Amalgam, Xenon or LED lamp technology). |
| 104 | Easy Access Panel | A quick panel access has been included with the invention to allow for |
| | | easy maintenance and to quickly swap a battery. |
| 201 | Ergo Handle 1 | Ergonomic top handle placed in an easy position for the operator to lift |
| | | up the stairs. |
| 202 | Camera Sensor | Camera to aid with autonomous movement and safety. |
| 203 | Lidar Sensor | Device to detect distance to aid with autonomous movement and |
| | | safety. |
| 204 | Touch Screen | Single touch screen to start or stop the device. |
| 205 | Master Power | Master power disconnect switch, to disconnect all electronics from |
| | | battery. |
| 206 | Sonar Sensor | Device to detect distance to aid with autonomous movement and |
| | | safety. |
| 207 | Charge Receptacle | Quick connect charge receptacle to easily charge the battery of |
| | | invention. |
| 208 | Ergo Handle 2 | Ergonomic lower handle placed in an easy position for the operator |
| | | lifting from the lower stairs. |
| 301 | Ergo Handle 3 | Ergonomic top handle placed in an easy position for the operator to lift |
| | | up the stairs. |
| 302 | Camera Sensor | Camera to aid with autonomous movement and safety. |
| 303 | Touch Screen | Single touch screen to start or stop the device. |
| 304 | Lidar Sensor | Device to detect distance to aid with autonomous movement and |
| | | safety. |
| 305 | Ergo Handle 4 | Ergonomic middle handle placed in an easy position for the operator |
| | | lifting from the upper stairs. |
| 306 | Sonar Sensor | Device to detect distance to aid with autonomous movement and |
| | | safety. |
| 401 | Emergency Switch 1 | Front Emergency Switch to disconnect the movement and the UVC |
| | | lamps if required. |
| 402 | Emergency Switch 2 | Rear Emergency Switch to disconnect the movement and the UVC |
| | | lamps if required. |
| 403 | Safety Light | Safety warning light to indicate the system is powered, armed |
| | | (emergency switch not pressed) and is ready to start. |
| 501 | Robotics Control | The robotics control centre is the brain power behind everything. The |
| | Centre | board is connected to many sensors and to the electromagnetic drive |
| | | wheels that provide guidance to the robot. This board has multiple Wi- |
| | | Fi, Bluetooth, and LTE connections. |
| 502 | DC to AC Converter | This component converts the DC power from the batteries to AC |
| | | power, required for the electrical ballasts. |
| 503 | Electrical Ballast 1 | The ballast converts the standard AC power into the required voltage, |
| | | current and frequency required to power the lamps. |
| 504 | Electrical Ballast 2 | The ballast converts the standard AC power into the required voltage, |
| | | current and frequency required to power the lamps. |
| 505 | Battery | A highly efficient battery is used to power the invention. |
| 601 | Electro Magnetic | To minimize space and weight, electromagnetic drive wheels have |
| | Drive Wheels | been included with the design. |
| 602 | Castor Wheels | The castor wheels are used to support the robot from the opposite end |
| | | of the drive wheels. |

### Alternate Embodiments

As the technology evolves, we are considering the utilization of alternate UVC producing technologies. UV-LEDs are growing in popularity (typically producing light around 280 nm), unfortunately their efficiency is not great enough for this application to be possible at the time of this writing.

### REFERENCES

Bolton, J. R., 2010. “Ultraviolet Applications Handbook”, 3rd, ICC Lifelong Learn Inc., 628 Cheriton Cres., NW, Edmonton, AB, Canada T6R 2M5.

1. A UVC disinfection device designed for mass transportation, comprising of:
unique vertical UVC lamps with an open cage configuration to minimize light absorption, maximize light emission, enable quick lamp change, and to protect lamps from bumps and other vibrations; and
require zero mechanical adjustments and/or human intervention prior and while in operation. (i.e., no expandable extremities); and
is easily portable, including narrow dimensions, light weight and handle placements designed to easily fit through the required tight spaces and stairways of mass transportation vehicles (doors, aisles, lavatory, cockpit etc).

2. The UVC disinfection device designed for mass transportation of claim 1 consists of autonomous features, including the following:
detect the aisle (or narrow space) width; and
detect the direction to move; and
navigate through the aisles (or narrow spaces); and transition between different aisle sizes; and
operate without selecting any pre-configured maps or settings.

3. The UVC disinfection device designed for mass transportation of claim 1 includes a stationary mode in addition to the autonomous mode, enabling the device the ability to disinfect smaller enclosed spaces (washrooms, lunchrooms, offices, waiting areas) without any movement.

4. Connectivity: The device remains connected (including Wi-Fi, cellular and Bluetooth) prior, during and after operation, providing real time data for statistics, status, remote dashboards, and maintenance; and
remote operation through a personal computer, laptop, tablet, or mobile device as a secondary option for control.
